(19) United States
(12) Patent Application Publication
(10) Pub. No.: US 2024/0125763 A1
(43) Pub. Date: Apr. 18, 2024

(54) DEVICE TO PREDICT TYPE 2 DIABETES

(71) Applicant: Aadi GANNAVARAM, Potomac, MD (US)

(72) Inventor: Aadi GANNAVARAM, Potomac, MD (US)

(21) Appl. No.: 18/489,300

(22) Filed: Oct. 18, 2023

Related U.S. Application Data
(60) Provisional application No. 63/417,074, filed on Oct. 18, 2022.

(51) Int. Cl. G01N 33/497; G16H 50/20

(52) U.S. Cl. CPC G01N 33/497; G16H 50/20; G01N 2033/4975

(57) ABSTRACT

The present disclosure relates to a device comprising a unit having an interior aqueous environment surrounded by a lipid layer, wherein the lipid layer comprises an olfactory receptor, wherein the unit is attached to the device via a hydrophobic force, wherein the device is configured to capture a volatile organic compound (VOC) onto the olfactory receptor. Further, the present disclosure also relates to the method and device comprising an artificial olfactory sensing system.

### RELATED APPLICATION

This application claims priority from U.S. provisional application 63/417,074, filed on Oct. 18, 2022, and entitled as, “DEVICE TO PREDICT TYPE 2 DIABETES”, which is herein incorporated by the reference in its entirety.

### FIELD OF THE INVENTION

Embodiments of the present disclosure relate to a device to detect Volatile Organic Compounds (VOC) released from oral or gut microbiomes and the method thereof.

### BACKGROUND OF INVENTION

Diabetes is a chronic condition affects a third of all Americans. More than 122 million Americans are living with diabetes or prediabetes, which imposes an estimated cost of $327 billion in medical costs and lost productivity annually (1). Lifestyle changes are a key component of the diabetes prevention and have proven to be effective at preventing type 2 diabetes.

The earliest method to test diabetes dates as far back as 600 B.C, as Chinese, Indian, and Egyptian scientists noticed ants were drawn to the urine of diabetic individuals. Doctors would drink urine and gauge the sweetness of the taste to determine if the sugar content was abnormally high, indicating diabetes (2). However, the first clinical test was not introduced until 1841 when Dr. Karl Tommer performed acid hydrolysis on patients' urine samples, breaking up the sugars in the sample into monosaccharides before introducing a reactant. The reaction would indicate whether an exceedingly high level of sugar was present (3). In 1908, American chemist Stanley Benedict developed the Benedict reagent, a solution that changed colors depending on the level of simple carbohydrates (sugars) present in a urine sample mixed with the solution when the mixture was heated (4). The first semi-quantitative diabetes test called Clinistix to measure glucose was created in 1956 by Alfred Free of Miles Laboratories. These were glucose oxidase reagent strips that tested glucose in urine by changing colors when dipped into a urine sample (5). Then, in 1964, the Dextrostix reagent strips were released, which tested for glucose directly in the blood (6). This made detection of diabetes faster and easier, as it could be done through finger prick samples. This evolved into the random blood glucose test that is widely used today.

Therefore, currently, there are two methods of detecting type 2 diabetes (referred to as diabetes henceforth). The first method is a random blood sugar test as described above. A random blood-sugar test tells physicians the amount of glucose circulating a person's body at a certain point in time. This measurement is made by taking a blood sample from the patient. From this, the physicians can determine a patient's chance for diabetes by comparing the result to the threshold for diabetes, 200 milligrams of glucose per deciliter (7). Comparing the patient's result to the threshold is important as the amount of glucose provides signs of how much glucose is not being used due to a lack of insulin. By the time elevated blood glucose levels are detected, it is too late to make preventative lifestyle changes.

The second method is a glycated hemoglobin, or A1C test. This method tests the attachment of glucose to hemoglobin in the blood. An A1C test measures the average amount of glucose in a person's bloodstream over the past 90 days as a percentage. This test is often used as a secondary test after a random blood-sugar test in order to verify the original diagnosis (8). Two different tests resulting in a 6.5% or higher warrants a diabetes diagnosis. Of the current methods, there are numerous limitations. For the random blood-sugar test, the main limitations include physiological and medication-related factors (8). The most significant physiological limitation for a random blood-sugar test is that peripheral blood perfusion can be lost due to hypotension. This may affect the result of the test as peripheral hypoperfusion may result in a lower glucose value compared to venous blood. For medication-related factors, the main lamination is that blood-sugar tests can be interfered with by common drugs such as Acetaminophen. Beyond medical limitations, blood-sugar tests require multiple tests in order to determine a more accurate diagnosis. The most significant limitation is that an A1C assay is unreliable and cannot be used on many types of people. Underlying conditions such as pregnancy, smoking, uremia, chronic anemia, etc, are responsible for misleading A1C data. These blood tests, to monitor diabetes, are performed during health checkups that many uninsured, at-risk people cannot afford. Recent discoveries describing the causal relationship between gut microbiota and the development of diabetes created possibilities to predict the onset of diabetes sooner than blood chemistry tests.

The human mouth is heavily colonized by microorganisms, with over 700 species identified (9). Collectively these organisms are called oral microbiome and play a role in systemic health through immune regulation, nutrition absorption and metabolism (10). Growing evidence suggests that the oral microbiome plays an important role in obesity and diabetes. It must be noted that many studies identified oral microbes primarily by metagenomic analysis. The analytes were detected using a technique called gas chromatography, where a sample is dissolved and vaporized before each component is analyzed (11).

Companies such as Viome offer fecal-sample tests to determine the gut microbiota (12). However, current methods of assessing gut microbes through metagenomic analysis of fecal samples to evaluate diabetes risk are yet to be clinically validated. The main limitation of these methods is that these are expensive, performed at specialized laboratories, are not generally covered by insurance and are yet to make an impact on diabetes prevention. This means that only affluent people can effectively utilize the potential of this method.

Therefore, there is a long felt need for a device that is inexpensive, sensitive, accessible and provides the results with great accuracy.

### SUMMARY OF INVENTION

The present application provides a device and a method to detect Volatile Organic Compounds (VOC) released from oral microbiomes or gut microbiomes with great accuracy.

In an embodiment, a device comprising a component comprising a wall having an outer layer and an inner layer, wherein the inner layer comprises a hydrophobic layer comprising an unit having a lipid layer surrounding an environment comprising an aqueous environment, wherein the lipid layer comprises an olfactory receptor configured to capture one or more volatile organic compounds (VOCs), wherein the device is configured to detect one or more VOCs.

In an embodiment, capture of one or more VOCs is configured to produce a signal.

In an embodiment, the unit and the hydrophobic layer of the inner layer interact using a hydrophobic bond.

In an embodiment, the hydrophobic bond is configured to form a space between the unit and the hydrophobic layer such that the space is configured to allow passage of the one or more VOCs to the olfactory receptor present on the unit.

In an embodiment, the device is configured to detect one or more VOCs with a sensitivity within a range of about one part per million to about one part per billion.

In an embodiment, the sensitivity is about one part per billion.

In an embodiment, one or more VOCs comprise molecules released from a microbiome of a user using the device.

In an embodiment, the microbiome comprises gut microbe and/or oral microbiome.

In an embodiment, the microbiome includes Actinobacteria and/or Prevotella.

In an embodiment, the lipid layer comprises a lipid bilayer.

In an embodiment, component comprises a slit comprising a microslit having the unit.

In an embodiment, the device is configured to detect onset of a diabetes.

In an embodiment, a method to detect one or more volatile organic compounds (VOCs) comprising: entering of one or more VOCs into a slit of a device; attaching one or more VOCs to a unit having a lipid layer surrounding an environment comprising an aqueous environment, wherein the lipid layer comprises an olfactory receptor; capturing of the one or more VOCs by the olfactory receptor; generating one or more ionized molecule from the capture of one or more VOCs; and producing an electrophysiological signal.

In an embodiment, one or more VOCs comprise one or more microbial VOCs.

In an embodiment, the method further comprises analyzing the VOCS by employing a deep learning algorithm.

In an embodiment, the method is configured to detect a diabetic condition of an user.

In an embodiment, a system comprising an artificial olfactory sensing system comprising: a unit having a lipid layer surrounding an environment comprising an aqueous environment, wherein the lipid layer comprises an olfactory receptor; a transistor to capture an ionized molecule to generate an electrophysiological signal; a monitor to display the electrophysiological signal in an quantitative and an qualitative manner; wherein the system is configured to detect one or more Volatile Organic Compounds (VOCs).

In an embodiment, VOCs comprises at least one of (S)-2-hydroxypropanoic acid, heptylhydroperoxide, 2,3-dihydroxypropanal, nonanoyl chloride, dodecanal, (Z)-2-nonenal, 4,5-dimethyl, -3(2H)-isoxazolone, (Z)-2-decenal, trichloro acid 3-tridecyl ester, levoglucosan, 4-(dimethyl amino)-3-methyl-2-butanone, 4-methyl-1-butene-1,1-pentanoic acid ester, diethylphthalic acid, 1-chloro-8-heptadecene, pentadecanoic acid, 1,2-benzenedicarboxylic acid butyldecyl ester, nonanal, 1-butanol, 3-hydroxy-2-butanone, hexanol, 2-pentanone, tetrahydrofuran, 2-methylpyrazine, (E)-2-nonenal.

In an embodiment, the ionized molecule is negatively charged.

In an embodiment, the system comprises a deep learning algorithm configured to detect one or more VOCs.

### BRIEF DESCRIPTION OF THE FIGURES

The drawings described herein are for illustrative purposes and are not intended to limit the scope of the present application.

FIG. 1 shows a schematic view of the VOC detection system.

FIG. 2 shows the overall schematic view of a device.

FIG. 3 shows the schematic view of VOC receptors.

### DETAILED DESCRIPTION

### Definitions and General Techniques

For simplicity and clarity of illustration, the drawing figures illustrate the general manner of construction, descriptions, and details of well-known features. Techniques may be omitted to avoid unnecessarily obscuring the present disclosure. Additionally, elements in the drawing figures are not necessarily drawn to scale. For example, the dimensions of some of the elements in the figures may be exaggerated relative to other elements to help improve understanding of embodiments of the present disclosure. The same reference numerals in different figures denotes the same elements.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements but may include other elements not expressly listed or inherent to such process, method, system, article, device, or apparatus.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments of the apparatus, methods, and/or articles of manufacture described herein are, for example, capable of operation in other orientations than those illustrated or otherwise described herein.

No element, act, or instruction used herein should be construed as critical or essential unless explicitly described as such. Also, as used herein, the articles “a” and “an” are intended to include items and may be used interchangeably with “one or more.” Similarly, the words “comprise”, “comprises”, and “comprising” are to be interpreted inclusively rather than exclusively. Likewise, the terms “include”, “including” and “or” should all be construed to be inclusive, unless such a construction is clearly prohibited from the context. The term “example” used herein, particularly when followed by a listing of terms, is merely exemplary and illustrative and should not be deemed to be exclusive or comprehensive.

Additionally, further, to be noted, when the elements and the embodiments thereof of the present application are introduced, the articles “one”, “the” and “said” are intended to represent the existence of one or more elements. Unless otherwise specified, “a plurality of” means two or more. The expressions “comprise”, “include”, “contain” and “have” are intended as inclusive, there may be other elements besides those listed.

In addition, in the drawings, the thickness and area of each layer are exaggerated for clarity. It should be understood that when a layer, a region, or an element is referred to as being “on” another part, it is meant that it is directly on another part, or there may be other elements in between. In contrast, when a certain element is referred to as being “directly” on another part, it is meant that no other element lies in between.

Furthermore, as used herein, the term “set” is intended to include items (e.g., related items, unrelated items, a combination of related items, and unrelated items, etc.), and may be used interchangeably with “one or more.” Where only one item is intended, the term “one” or similar language is used. Also, as used herein, the terms “has,” “have,” “having,” or the like are intended to be open-ended terms. Further, the phrase “based on” is intended to mean “based, at least in part, on” unless explicitly stated otherwise.

The present invention may be embodied in other specific forms without departing from its spirit or characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

As defined herein, “approximately” can, in some embodiments, mean within plus or minus ten percent of the stated value. In other embodiments, “approximately” can mean within plus or minus five percent of the stated value. In further embodiments, “approximately” can mean within plus or minus three percent of the stated value. In yet other embodiments, “approximately” can mean within plus or minus one percent of the stated value.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular.

The methods and techniques of the present invention are generally performed according to conventional methods well known in the art and as described in various general and more specific references that are cited and discussed throughout the present specification unless otherwise indicated.

The following terms and phrases, unless otherwise indicated, shall be understood to have the following meanings.

Exemplary embodiments will now be described more fully with reference to the accompanying drawings.

The term ‘device’ herein refers broadly to a thing made or adapted for a particular purpose, such as a piece of mechanical or electronic equipment. In an embodiment, the device herein may be a handheld device and used to detect the Volatile Organic Compounds (VOCs).

The term ‘unit’ herein refers to a single and complete-by-itself object but can also be part of a larger or more complex whole. In an embodiment, the unit could be an aqueous droplet that constitutes a complete mechanism of perceiving the VOCs released from the microbiomes. In an embodiment, the unit mimics the olfactory mechanism present in insects.

The term ‘aqueous environment’ herein refers to a surrounding containing water or the like molecules that are compatible with water. In an embodiment, aqueous environment could be an enclosed system or confined area defined by a layer and having water such as but not limited to droplet or drop. Under normal conditions, it is assumed that presence of water in the aqueous environment causes hydrophobic residues to congregate near the centre of the protein and be shielded from contact with water, whereas hydrophilic residues are exposed on the proteins surface.

The term ‘lipid layer’ herein refers to a membrane or flat sheets of lipids that form a continuous barrier around a thing, that may or may not be interspersed by some other groups such as but not limited to proteins, phosphates or carbohydrates. In an embodiment, the lipid layer is around an aqueous droplet. In an embodiment, the lipid layer could be a single layer or bilayer or multiple layers. In another embodiment, the lipid layer has receptors. These receptors may be of natural or synthetic origin. In yet another embodiment, the receptors are olfactory receptors.

The term ‘olfactory receptor’ also known as odorant receptor or like, are chemoreceptors expressed in a membrane and responsible for the detection of odorants (for example, compounds that have an odour) which give rise to a sense of the smell. An olfactory receptor can display affinity for a range of odour molecules or a specific odor molecule. For example: a single odorant molecule may bind to a number of olfactory receptors with varying affinities, which may depend on the physio-chemical properties of molecules. An example of physio-chemical property of the molecule could be but not limited to molecular volumes, molecule structure, etc. In an embodiment, odour receptors provide a system to distinguish different odours via detection of different odour molecules. In an embodiment, individual odour receptor could be tuned to be activated by several similar odorant structures.

The water-hating molecules are also called as hydrophobic groups.

The term ‘hydrophobic bond’ herein refers to an attraction between water-hating molecules. The attraction between water-hating molecules could bring change in physical phenomena, for example: hydrophobic regions of proteins aggregate to form globules. In some embodiment, hydrophobic forces may assist in formation of a channel, etc. In an embodiment, interior region of the wall of the device is coated with a hydrophobic material. The hydrophobic material could generate hydrophobic force due to interaction of the wall and the lipid layer of the unit.

The term ‘capture’ herein refers to attachment or interaction of a substrate (molecule) with a ligand. Attachment of interaction of the molecule to its receptor could be due to formation of a chemical bond between the molecule and its receptor. In an embodiment, capture could be an interaction of VOC molecule with its receptor. The receptor could be an olfactory receptor.

In yet another embodiment, the interaction or capturing of the molecule to its receptor could produce a signal. In an embodiment, transistor captures an ionized molecule to generate an electrophysiological signal. In an embodiment, the signal could be measured and/or detected.

The term ‘volatile organic compound/s’ or VOC or VOCs herein refers to organic compounds which are volatile at a room temperature. VOCs are originated from a variety of sources. In an embodiment, VOCs could be originated from microbes preferably but not limited to oral or gut microbiomes. These VOCs are referred to as microbial volatile organic compounds or mVOCs.

The term ‘slit’ refers to a space through which a ray of light can pass through it. When slit has a micron range, then it is called as microslit.

The term ‘onset of diabetes’ signifies starting point of a diabetes.

The term ‘oral or a gut microbiome’ herein refers to the microbiome that resides in the oral or the alimentary canal or a portion thereof, especially in an intestine or a stomach. In an embodiment, the oral or gut microbiome or microbiota herein relates to a particular disease but not limited to obesity or diabetes.

The term ‘user’ herein refers to a subject that includes mammals such as human.

The term ‘slit’ herein refers to a long cut or opening or recess in the device. These opening could be of a dimension of 1 micron, 10 micron, 50 micron, 100 micron, 1000 micron or more, or within any range of the mentioned values. In an embodiment, slits in the device are in an array configuration. In another embodiment, slits are of micron size and are microslits.

The term ‘ionized molecule’ herein refers to a molecule that acquires a negative or positive charge by gaining or losing electrons, often in conjunction with other chemical changes.

The term ‘deep learning algorithm’ or ‘a deep learning algorithm’ herein refers to an algorithm that dynamically run data through one or several layers of networks such as decision-making networks that are pre-trained to serve a task. Data could be passed through one or more layers of the networks to processes the data. In an embodiment, network could be a neural network or an artificial neural network.

The term ‘artificial olfactory sensing system’ herein refers to a replica of an olfactory sensing system employing one or more components made manually.

The term ‘transistor’ herein refers to a semiconductor device that regulates or controls current or voltage flow to amplify or generate electrical signals.

The term ‘monitor’ herein refers to an output device that displays information either in a pictorial or a text form or any form known to a person skilled in the art. In an embodiment, monitor comprises a visual display, some circuitry, a casing, and a power supply. In an embodiment, monitor could be a computer device.

The foregoing description of the embodiment has been provided for purpose of illustration and description. It is not intended to be exhaustive or to limit the application. Even if not specifically shown or described, individual elements or features of a particular embodiment are generally not limited to that particular embodiment, are interchangeable when under a suitable condition, can be used in a selected embodiment and may also be varied in many ways. Such variations are not to be regarded as a departure from the application, and all such modifications are included within the scope of the application.

In an embodiment, the artificial olfactory sensor provided is a sensitive and inexpensive sensor modelled on insect olfactory receptors that detect Volatile Organic Compounds (VOCs) characteristic of oral microbiome with great accuracy. In another embodiment, by monitoring the VOCs released by specific oral microbiome, the onset of diabetes can be predicted.

In an embodiment, the device is a handheld device that enables inexpensive and frequent sampling of oral microbiome at home and enables or suggests preventative lifestyle changes.

In an embodiment, there is a direct link between pathogenic bacteria (such as P. gingivalis and A. actinomycetemcomitans) and glycemic control and diabetes risk. Higher abundance of the phylum Actinobacteria and almost all taxa in this phylum are associated with a decreased risk of diabetes; and an abundance of Gemellaceae in the phylum or Firmicute is associated with an elevated risk of diabetes.

In another embodiment, the ratio of Firmicutes to Bacteroidetes increased in type 2 diabetes and these patients presented significantly higher numbers of Neisseria, Streptococcus, Haemophilus, and Pseudomonas genera, and lower numbers of Acinetobacteria compared with healthy controls.

In an embodiment, oral microbes release volatile organic compounds (VOCs) characteristic of the species of the microbes.

In an embodiment, the device targets VOCs for detection with an electronic nose as a surrogate marker of the onset of diabetes based on the presence or absence of certain microbes. Thus, the device circumvents the need for expensive metagenomic analysis and allows easy monitoring of the oral microbiome as a prognostic biomarker by switching to VOCs as analytes.

In an embodiment, the first key insight of the present technology is that VOCs released by oral microbes can predict the onset of diabetes. The association between certain microbes and the risk of onset diabetes has already been explored. Building on these clinical studies in large cohorts, the device targets to monitor changes in the diversity of the oral microbiota by analyzing VOCs that are characteristic of microbial species previously identified to be associated with the development of diabetes.

In an embodiment, Actinobacteria produced the C1 to C5 (hydrocarbon) hetero-VOCs. These include oxygenated nitrogen and sulfur containing compounds, alcohols, and esters.

In an embodiment, the VOCs released by specific microbes are required to make an accurate prediction of diabetes onset.

In an embodiment, the device uses an electronic nose to detect VOCs, specifically, modeled on an insect olfactory-receptor-based sensor.

In an embodiment, there are insect cells that contain olfactory receptors (ORs) on their surfaces that detect VOCs. The concept of mimicking insect olfactory receptors (the way insects “smell”) has been exploited to detect VOCs such as Octenols to 1 part per billion sensitivity by constructing a synthetic Olfactory trapped in a lipid bilayer.

In another embodiment, Octenol is not the relevant VOC to predict an early onset of diabetes thus a hybrid sensor that can detect specific analytes related to diabetes is needed. Such a reconstituted sensor will have a longer shelf life and enable simplified detection.

In an embodiment, the device has high levels of accuracy in VOC detection by employing a deep learning algorithm in its receptor. Preliminary evidence for such accuracy, i.e., the aforementioned phylum Actinobacteria, has been established.

In an embodiment, to identify the critical oral microbes that mediate development of diabetes, machine learning algorithms such as deep learning may be used following analysis of VOCs. Machine learning refers to computer algorithms that improve in their accuracy of identifying and classifying data. Deep learning is a type of machine learning designed to mimic the human brain, as it continues to improve accuracy with massive data sets such as types of oral microbes.

In an embodiment, the deep learning algorithm can be used to identify the oral microbes that help predict whether or not a person is at risk for developing diabetes.

In an embodiment, once the VOCs of target oral microbes are identified, the olfactory receptors are engineered to identify specific VOCs. The multiplex Olfactory Receptor based sensors in the units, with a deep learning algorithm, are designed to detect VOCs from oral microbes with the highest risk association for diseases. There may not be a naturally occurring insect olfactory receptor that can detect VOCs of interest.

In an embodiment, computational protein design approaches to design proteins de novo with desired activity can be used.

In an embodiment, during the engineering phase, other things are necessary, such as maintaining the stability of the receptor structure, preventing the receptor structure from being saturated, calibrating the receptor, calibrating the device or recalibrating and regeneration of receptors towards real-time, continuous measurement either manually or by the software.

In an embodiment, further study into these issues is required to achieve the breakthrough, but some ideas include a microfluidics system to control the fluid around the receptors, and nanoscale constructs in the structure of the receptors.

In an embodiment, the final step to ensure that the technology is clinically applicable on a more formal basis is to evaluate the accuracy in longitudinal studies to confirm the prediction, using the device.

In an embodiment, towards controlling diabetes, three alternative solutions before settling on the device are considered.

In an embodiment, the first method or solution for controlling diabetes is to create a probiotic containing Prevotella copri for people with unhealthy gut microbiota since Prevotella copri is associated with lower diabetes risk. The purpose of the probiotic is to restore gut microbiota and achieve hypoglycemic effects.

However, there is a risk in introducing new microbes to an unhealthy body which might cause microbial imbalance in the gut, e.g., Dysbiosis. Dysbiosis is detrimental as it can cause inflammatory bowel disease, inflammatory bowel syndrome, cancer, obesity, and other harmful conditions.

In an embodiment, the second method or solution for controlling diabetes is to detect diabetes by metagenomic sequencing of gut microbes. Gene sequencing is accomplished by taking fecal samples from the human subjects and assessing the microbes in the samples by comparison of reference standards. This method has numerous limitations. Companies such as Viome are already analyzing gut microbes through gene sequencing of fecal samples. The causal relationship between gut microbial diversity and diabetes risk is not fully established. Therefore, such methods still need further development. More importantly, cataloging the gut microbes by metagenomic sequencing of fecal samples is a slow and costly process. Further, since this is too sophisticated to be performed as an at home test, the at-risk population are not going to access this test.

In an embodiment, the third method or solution for controlling diabetes is based on an observation that glucagon-like-peptide (GLP-1) shows antidiabetic effects. GLP-1 is secreted by the intestinal cells and can decrease post-meal blood sugar by enhancing the biosynthesis and secretion of insulin.

In an embodiment, identifying the predominant gut microbe and engineering it to secrete GLP-1 peptide as an endogenous regulator of insulin production, is considered. The secretion of GLP-1 peptide would prevent development of diabetes. There are numerous limitations to this method as well. First, it is not clear if the predominant gut microbes are amenable to recombinant DNA engineering to secrete sufficient quantities of insulin. Further, introducing engineered gut microbes would require clinical validation.

In an embodiment, ultimately, the method selected is to perform a prediction of diabetes long before any existing methods can do so by analyzing oral microbes through the use of an at-home hand-held device with a sensitive sensor that can detect the presence of oral microbes by measuring VOCs emanated by the oral microbes.

In an embodiment, this method has many advantages over the previously considered methods. First, the selected method provides for prediction of the onset to patients rather than diagnosis. Prediction is preferable to treatment since it allows for preventative actions recommended by the CDC. Second, compared to the second considered method (metagenomic sequencing of gut microbes), the use of the olfactory receptor-based sensors and oral microbes means that results can be acquired faster and at a lower price.

In an embodiment, while both oral microbes and gut microbes have been proven to be effective towards identifying diabetes, gut microbe detections through gene sequencing requires more time to complete as the whole mixture of microbes must be analyzed to find a genetic-material-match. In comparison, the olfactory receptor-based sensors arranged in an array of 2D grids can perform a multiplex detection and generate an electrical signal for each VOC detected. From the VOC signals, the heterogeneity of oral microbes will be identified that can be used for prediction if alterations are detected over time.

In an embodiment, overall, the claimed method herein is more efficient and has the benefit of ease of use. Due to the ease of use, it may be widely and frequently used by the at-risk population and may help in better management of diabetes and achieve public health goals with respect to the National Diabetes Prevention Program.

In an embodiment, the most significant positive outcome of the device is that it will help in minimizing the harmful effects of diabetes by allowing monitoring of surrogate markers quickly and easily. Since the discovery of the connection between dysbiotic gut/microbiome and the development of diabetes, identification of prognostic markers that can foretell the onset has been the holy grail of diabetes prevention. Currently there are no methods that can reliably detect the onset of diabetes, only to detect prediabetes. By the time prediabetes is detected, the point of no return may have already been reached. Gut/oral microbiome analysis attempting to advance the prediction window depends on expensive sequencing and chromatographic techniques. The elective procedures currently commercially available that assess the gut/oral microbiome are often costly and inaccessible to the at-risk population.

In an embodiment, the device combines the power of biologically inspired olfactory sensors that can detect a single VOC molecule with the predictive potential of the altered oral microbiome in the onset of diabetes.

In an embodiment, the device will enable frequent and at-home measurement of VOCs characteristic of diabetes onset. Detection of early signals will make adoption of the CDC-recommended Diabetes Prevention Programs far easier in the at-risk population.

In an embodiment, the device is intended to reduce the necessity of visits to the doctor with an easy, at-home method to predict diabetes. This can allow people to substitute periodic checkups entirely.

In an embodiment, the non-invasive easy to use device, that can be produced at scale at a low cost, may revolutionize the management of diabetes. A future can be foreseen where affordable devices like ours will be a key element of detection and prevention of diabetes.

In an embodiment, the device further comprises a means to generate an electrophysiological signal after the capture of the VOC onto the olfactory receptor.

In an embodiment, the units in the device are arranged in an array or a grid configuration.

In an embodiment, the hydrophobic force forms a channel between the units.

In an embodiment, the device is configured to detect the onset of diabetes.

In an embodiment, the channel allows flow of the VOC to be captured at the olfactory receptor present in the unit.

In an embodiment, the VOCs are released from an oral or a gut microbiome of a user.

In an embodiment, the microbiome includes Actinobacteria and Prevotella.

In an embodiment, the device further comprising a slit or an array of slits.

In an embodiment, the hydrophobic force is between the interior hydrophobic wall of the device and the unit.

In an embodiment, the lipid layer comprises a lipid bilayer.

In an embodiment, the slit or the array of slits comprises microslits.

In an embodiment, the microslits comprising olfactory receptors to detect different VOCs.

An embodiment relates to a method to detect volatile organic compounds (VOCs) comprising: entering of VOCs into a slit; attaching of VOCs to a unit having an interior aqueous environment surrounded by a lipid layer, wherein the lipid layer comprises a receptor; capturing the VOCs by the receptor; generating an ionized molecule after capturing; releasing of the ionized molecule to deliver an electrophysiological signal.

In an embodiment, the method further comprises analyzing the VOCs by employing a deep learning algorithm.

In an embodiment, the volatile organic compounds comprise microbial volatile organic compounds.

In an embodiment, the method helps in detecting a pre-diabetic living being.

In an embodiment, one or more phylum bacteria related to diabetes, selected from the group consisting of Proteobacteria, Actinobacteria, Verrucomicrobia, and Bacteroidetes, and Tenericutes Thermus, Fusobacteria, Chloroflexi, Cyanobacteria, TM7, Euryarchaeota, proteobacteria, Betaproteobacteria, Actinobacteria, Gammaproteobacteria, Clostridia, Verrucomicrobiae, and Bacteroides.

In an embodiment, the present invention relates to the device and a method for screening a sample (for use in the treatment of a subject suffering from a disease or condition) selected from the group consisting of Aeromonadales, Deinococcales, Cytophagales, Rhizobiales, Neisseriales, (Fusobacteriales), Sphingobacterials, Sphingomonadales, Pseudomonadales, Rhodospirillales, Flavobacteriales, Fusobacterium, Fusobacteria, Fusobacteria, Rhodocyclales, Rhodobacterales, Gemellales, Caulobacterales, Actinomycetales, Xanthomonadales, Alteromonadales, Pasteurellales, Bacillales, Burkholderiales, Lactobacillales, Clostridiales, Clostridiales, Verrucomicrobiales, and Bacteroidales, Stramenopiles, Pseudomonadales, Coryobacterium species.

In addition to the screened samples, other samples that can be screened consist of aeromonadaceae, Methylobacteriaceae, Rhizobiaceae, Bradyrhizobiaceae, Halomonadaceae, Cytophagaceae, Neisseriaceae, Fusobacteriaceae, Sphingomonadaceae, Weeksellaceae, Moraxellaceae, Aerococca, and so on. The samples could also consist of, but not limited to, Aerococcaceae, Pseudomonadaceae, Micrococcaceae, Propionibacteriaceae, Intrasporangiaceae, Gemellaceae.

In an embodiment, different VOCs related to halomonas, Methylobacterium, Neisseria, Fusobacterium, Kaistobacter, and Agrobacterium, such as Porphyromonas, Cupriavidus, Acinetobacter, Pseudomonas, Chryseobacterium, Sphingomonas, Rothia, Micrococcus, Enhydrobacter, Propionibacterium, Brevibacterium, Corynebacterium, Lautropia, Paracoccus, Staphylococcus, Hemophilus, Haemophilus, Catenibacterium, Anaerococcus, Prevotella, Actinomyces, Veillonella, Citrobacter, Enterococcus, Streptococcus, Dialister, Bacillus, Lactobacillus, Bifidobacterium, Faecalibacterium, Parabacteroides are detected by the device. Also detected by the device is Paraprevotella, Akkermansia, Ruminococcus, Adlercreutzia, Butyricimonas, Odoribacter, Coprococcus, Anaerostipes, Blautia, Bacteroides, and Epulopiscium, Rhizobium and Cupriavidus, Acinetobacter, Pseudomonas, Lactobacillus, Citrobacter, Enterococcus, Paracoccus, Klebsiella.

Another embodiment relates to a device comprising an artificial olfactory sensing system comprising:
a unit having an interior aqueous environment surrounded by a lipid layer, wherein the lipid layer comprises an olfactory receptor;
a transistor to capture an ionized molecule to generate an electrophysiological signal;
a monitor to display the signal in a quantitative and a qualitative manner.

In an embodiment, the unit is configured to capture Volatile Organic Compounds (VOCs).

In one embodiment, the VOCs comprises (S)-2-hydroxypropanoic acid, heptylhydroperoxide, 2,3-dihydroxypropanal, nonanoyl chloride, dodecanal, (Z)-2-nonenal, 4,5-dimethyl. -3(2H)-isoxazolone, (Z)-2-decenal, trichloro acid 3-tridecyl ester, levoglucosan, 4-(dimethylamino)-3-methyl-2-butanone, 4-methyl-1-butene-1, 1-pentanoic acid ester, diethylphthalic acid, 1-chloro-8-heptadecene, pentadecanoic acid, 1,2-benzenedicarboxylic acid butyldecyl ester, nonanal, 1-butanol, 3-hydroxy-2-butanone, hexanol, 2-pentanone, tetrahydrofuran, 2-methylpyrazine, and (E)-2-nonenal.

In an embodiment, olfactory receptors are insect olfactory receptors. In another embodiment, the insect cells are moth-derived cells.

In an embodiment, the insect cell expresses a fluorescent protein that emits fluorescence based on a change in intracellular ion concentration. The signal is fluorescence emitted by the fluorescent protein, based on a change in intracellular ion concentration due to binding of an olfactory receptor protein and a volatile organic compound of biological origin.

In an embodiment, the device detects a plurality of different volatile organic compounds.

In an embodiment, the amount of the biogenic volatile organic compound detected by the device is according to, but not limited to, the ambience temperature, sample area.

In an embodiment, if the amount of the biologically derived volatile organic compound in the sample collected from the subject is large compared to the amount of the biologically derived volatile organic compound in the control sample, it is possible that the patient has a disease. In another embodiment, when the amount of the biologically derived volatile organic compound of the sample collected from the subject is equal to or smaller than the amount of the biologically derived volatile organic compound of the control sample, there is no disease.

In an embodiment, the present invention provides a method for detecting a volatile organic compound derived from a living body. Further, there is provided a method for inspecting a disease using the amount of a volatile organic compound derived from a living body, which is peculiar to the disease, detected by the detection method of the present invention as an index.

In an embodiment, the volatile organic compound derived from a living body which is a target of the detection method of the present invention is not limited as long as an olfactory receptor of an insect that binds to the volatile organic compound derived from the same living body is present. Here, the volatile organic compound is an organic compound having a property of easily evaporating at room temperature, a highly volatile organic compound having a boiling range of <0° C. to 100° C., a volatile compound having a boiling range of 50° C. to 260° C. It includes organic compounds and semi-volatile organic compounds having a boiling point range of 240° C. to 400° C. Preferred are volatile organic compounds having a boiling point range of 30° C. to 260° C.

In an embodiment, the molecular weight of the volatile organic compound is not particularly limited, but may be, for example, one having a molecular weight of about 10 to 400 u. Examples of the volatile organic compound derived from the living body targeted by the detection method of the present invention depends on the type of disease to be detected.

In an embodiment, insect cells can be used without limitation as long as insect olfactory receptors are functionally expressed.

In an embodiment, the insect cell may be a Drosophila-derived cell.

In an embodiment, the olfactory receptors are semi-synthetic or artificially produced.

In an embodiment, the olfactory receptor may be a G protein-coupled receptor or an ion channel type receptor.

In general, a particular type of olfactory receptor has specificity for particular VOCs. In an embodiment, plural kinds of olfactory receptors corresponding to different VOCs are there in the device. Further, the detection sensitivity as an odor sensor may be adjusted by adjusting the amount of olfactory receptors there in the device.

Signals emitted on the binding between olfactory receptors and biologically derived volatile organic compounds include, but are not limited to, intracellular ion concentration based on the binding between olfactory receptors and biologically derived volatile organic compounds. The intracellular ion concentration can be detected as a change in membrane potential, fluorescence intensity, etc. based on a change, etc. The signal emitted by the ORs can be detected by the measuring device, an optical sensor, or the like.

In an embodiment, a fluorescent protein may be expressed together with the olfactory receptor. For example, in the device, when a volatile organic compound of biological origin binds to an ion channel type olfactory receptor, ions such as calcium ions flow. Examples of fluorescent proteins include, but are not limited to, GCaMP3, GCaMP6s, aequorin, and the like.

In addition, a calcium ion-dependent fluorescent dye may be added to the olfactory receptor-expressing cells without coexpressing the fluorescent protein. By coexisting with a calcium ion-dependent fluorescent dye, the intracellular calcium ion concentration changes when the receptor binds to the target volatile organic compound derived from the living body, and fluorescence is emitted. As such, a fluorescent dye, for example, Fura-2, Fluo-3, Fluo-4 or the like, can be used.

In an embodiment, there are an array of slits or microslits capable to detect different VOCs. The number of slits or microslits in the device may be one, but the number of slits or microslits is not particularly limited and may be plural. Further, the device may have multiple units in a micros lit.

In an embodiment, the device is set up with a gas flow system that helps to detect VOCs at low concentrations by creating a gas flow channel. In another embodiment, gas flows at a uniform flow rate in the device.

In an embodiment, the size of the microslit is 1 μm, 2 μm, 3 μm, 4 μm, 5 μm, 10 μm, 15 μm, 20 μm, 25 μm, 50 μm, 75 μm, 100 μm, and so on.

In an embodiment, the size of the slit is in nanometres.

In an embodiment, there are microbeads in the droplet.

In an embodiment, the Olfactory receptor (OR) is a purified OR.

In an embodiment, the device is highly selective and sensitive and capable of molecular-level sensing of VOCs.

In an embodiment, the sensitivity of the device is defined as sensitive enough to detect the concentration of a VOC when present in a concentration of as low as ppm or ppb or ppt.

In an embodiment, the device is selective enough to differentiate enough between two VOCs. In another embodiment, the selectivity of the device is defined as the device or ORs are able to differentiate between two VOCs on the basis of their carbon length or functional groups or specific enough to differentiate between two VOCs on the basis of the presence of single or double or triple bond. Yet, in another embodiment, the device is chemoselective, regioselective or stereoselective.

In an embodiment, the sensitivity of the device is up to parts per million-level detection of VOCs. In an embodiment, the device can measure VOCs present at a concentration of 0.1 ppm, 0.2 ppm, 0.5 ppm, 1 ppm, 2 ppm, 5 ppm, 10 ppm, 15 ppm, 50 ppm, 100 ppm, and so on.

In an embodiment, the sensitivity of the device is up to parts per billion-level detection of VOCs. In an embodiment, the device can measure VOCs present at a concentration of 0.1 ppb, 0.2 ppb, 0.5 ppb, 1 ppb, 2 ppb, 5 ppb, 10 ppb, 15 ppb, 50 ppb, 100 ppb, and so on.

In an embodiment, the sensitivity of the device is up to parts per trillion-level detection of VOCs. In an embodiment, the device can measure VOCs present at a concentration of 0.1 ppt, 0.2 ppt, 0.5 ppt, 1 ppt, 2 ppt, 5 ppt, 10 ppt, 15 ppt, 50 ppt, 100 ppt, and so on.

In an embodiment, the fluorescence image or electrophysiological signal generated by the sensor or transistor may be passed to the determiner. The determiner has a processor, a semiconductor memory, and a peripheral circuit, and detects a target volatile organic compound derived from a living body based on the fluorescence image or electrophysiological signal output from the sensor.

In an embodiment, the sample is inhaled into the device or the sample can be prepared by any method as long as it can prepare a sample capable of detecting a volatile organic compound derived from a living body by an olfactory receptor. For example, the collected exhaled breath, urine, sweat, saliva, blood, and the like can be used as they are. Alternatively, for example, the sample can be prepared by dissolving and diluting the sample in an appropriate solvent.

In an embodiment, examples of the subjects to be tested by the detection method of the present invention include mammals including humans. For example, mammals other than humans include, but are not limited to, mice, rats, dogs, cats, rabbits, monkeys, horses, cows, and the like.

In an embodiment, in addition, a subject tested could be, a subject not suspected of having a disease such as diabetes, a subject suspected of having a disease, a subject already suffering from some disease, a subject after treatment or during treatment of a disease, etc.

In an embodiment, the device can detect a target volatile organic compound derived from a living body with high sensitivity of ppt level and high discrimination ability, and the sensor cells used in the detection method of the present invention can be used for a long period of time and have portability.

In an embodiment, in comparing the amount of biologically derived volatile organic compounds of a sample collected from a subject with the amount of biologically-derived volatile organic compounds of a control sample, if the amount of the volatile organic compound is large compared to the amount of the volatile organic compound derived from the living body of the control sample, it is determined that the patient has a disease or is likely to have a disease.

In an embodiment, although not limited, for example, the amount of the biogenic volatile organic compound of the sample collected from the subject is, for example, 1.2 times or more, preferably 1.5 times the amount of the biogenic volatile organic compound of the control sample. The above case, more preferably 2 times or more, further preferably 5 times or more, still more preferably 10 times or more, is judged to have a disease or a high possibility of having a disease.

In general, the variation can be easily confirmed by using the amount or expression pattern of the volatile organic compound derived from a living body in a sample derived from a healthy person or a patient as a control. In addition, in advance, the measurement result of the amount or expression pattern of the volatile organic compound derived from the living body in the sample derived from the healthy subject or the patient is prepared as a standard result, and it is determined whether there is a significant difference by comparing with the standard result. Such embodiments are also included in the method of the present invention.

The amount of the volatile organic compound derived from the living body of the sample may be compared by a value that correlates with the amount of the volatile organic compound derived from the living body (for example, a measured value of fluorescence intensity).

When using the amount of volatile organic compounds of two or more types of living body as an index, it may be determined as the total amount of the amount of volatile organic compounds of two or more types of living body-derived, volatile organic compounds of each living body. A comprehensive determination may be made based on the amount of compound (including the weight of any compound).

Further, when determining a disease using the amount of volatile organic compounds of two or more types of organisms as an index, the type of the disease is determined based on the coincidence with the occurrence pattern of the volatile organic compounds of the control sample.

In an embodiment, there is the variation results of the amount of one or more kinds of volatile organic compounds derived from a living body in a sample collected from a subject, in determination of a disease (presence or absence of disease), and in the type of a disease. The causative bacteria of infectious diseases and the degree of diseases (therapeutic effect, diagnosis of prognosis of diseases, etc.) is also analyzed.

In one embodiment, the ionized molecule is negatively charged.

In an embodiment, an artificial olfactory sensing system, comprising a unit which includes an olfactory receptor is manifested on a lipid layer or lipid bilayer, and a transistor; wherein the transistor includes: a substrate; a source region and a drain region which are formed in the substrate; and a gate electrode which is formed on the substrate between the source region and the drain region through a gate insulating film, wherein a first insulating film is formed on the gate electrode, wherein an electrolytic aqueous solution is disposed on the first insulating film, wherein a proton is adsorbed on the first insulating film, and wherein, when the olfactory receptor recognizes a VOC, positive ions flow into the transistor from an ion channel provided in the olfactory receptor and, as a result, the proton is dissociated from the first insulating film into the electrolytic aqueous solution, and the potential of the gate electrode is changed.

In an embodiment, there is an amplification of the molecular signal by the ion channel. In an embodiment, a single molecule signal is amplified to many million molecules per second.

In an embodiment, olfactory receptors have co-receptors attached to them in the device.

In an embodiment, the first insulating film is made of an aluminum oxide film of which a surface is porous and contains negative fixed charges.

In an embodiment, a hydroxyl group is bonded to an aluminum atom existing in the surface of the first insulating film.

In an embodiment, the first conductor film is a gold film, and wherein the first insulating film is a self-assembled monolayer which is made of molecules bonded through Au—S bonding in the gold film; wherein the molecule is made of a Br-bipyridine-derivative containing a thiol group or a sodium 2-mercaptoethanesulfonate.

In an embodiment, a stimulus time of the VOC or mVOC is measured from a continuous time of a potential change of the gate electrode which is caused when the olfactory receptor recognizes VOC or mVOC.

In an embodiment, a potential change of the gate electrode which is caused when the olfactory receptor recognizes the VOC is differentiated with time to measure a concentration of the VOC.

In an embodiment, a plurality of the units, a plurality of scanning lines, and a plurality of signal lines are included, wherein the unit is connected to one of the plurality of scanning and one of the plurality of signal lines, wherein plural kinds of units exist, and wherein the unit containing the same types of ORs is connected to the same scanning line among the plurality of scanning lines.

In an embodiment, the plurality of scanning lines is disposed to be crossed with the plurality of signal lines, respectively, and wherein the plurality of the units are disposed at intersections between the plurality of scanning lines and the plurality of signal lines.

In an embodiment, the transistor/s is/are configured with a deep learning algorithm.

In an embodiment, the device itself is a handheld cylinder with a tube for the user to blow through (FIG. 2). The VOCs travel through the tube, into a pump to push the gas through a regulator to maintain a constant flow of gas, and then through the arrays of microslits pictured in FIG. 2, where the VOCs are detected. The resulting electrophysiological signals are then transmitted to a monitor where analysis and predictions can be done.

In an embodiment, the olfactory receptors in the biohybrid sensor mechanism must be engineered to detect a variety of target VOCs. Each OR will be specific to a certain VOC (FIG. 3).

In an embodiment, the device minimizes harmful effects of diabetes by allowing for quick and easy monitoring of a surrogate marker resulting in a prognosis. Thus, a patient is able to change their lifestyle in order to prevent a possible risk of diabetes.

In an embodiment, the device is easily accessible and allows frequent measurements of VOCs characteristic of diabetes onset. Furthermore, the device will be affordable and space effective, meaning at-home testing is possible and local clinics can access these instruments as well.

In an embodiment, there is increased accuracy and legitimacy to results as the device provides accurate representation of oral microbes in a patient through the combination of biologically inspired olfactory sensors that can detect a single VOC molecule with the prognostic potential of the altered oral microbiome in the onset of diabetes. This allows a patient's condition to be accessed and an accurate prognosis can be acquired.

### Example 1

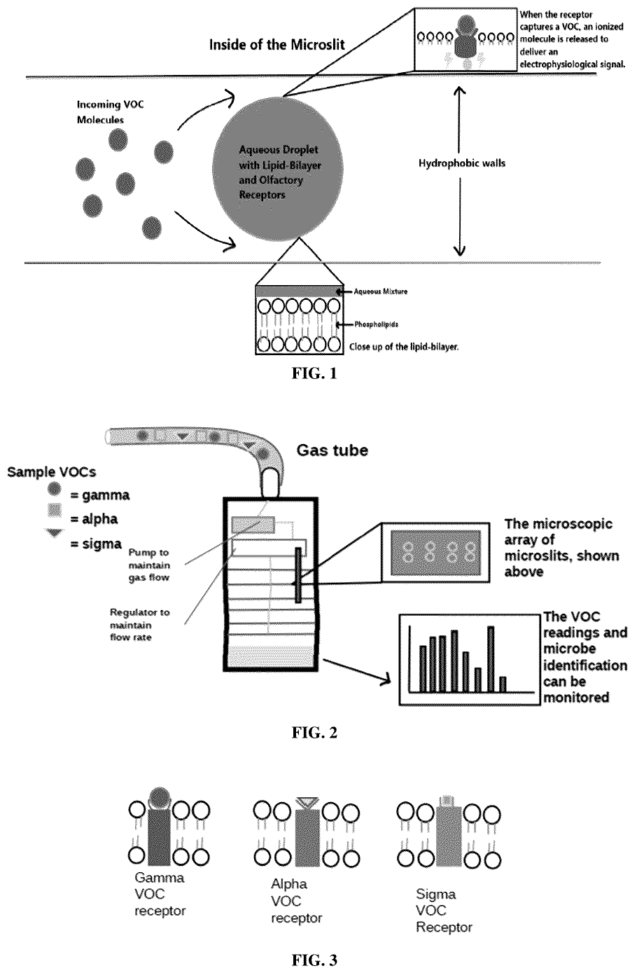

A spherical, aqueous droplet with a lipid-bilayer exterior analogous to a living cell forms the backbone of the detection system. Similar to an insect cells that contain ORs, the droplet is engineered to contain ORs on its surface in the lipid bilayer (see FIG. 1). Each aqueous droplet is inserted into a microslit with hydrophobic interior walls to maintain stability. The hydrophobic force also creates small channels where the droplet's surface and microslit walls repel and thus hold the droplet stable. The VOC molecules that a human subject exhales into the device travel through the channels are forced to come in contact with the surface ORs, where they are received, and an ionized molecule is released from the droplet that creates an electrophysiological signal. The microslits, measuring few microns wide each, are arranged in arrays, on a grid inside the device, where the VOC molecules pass through to maximize the detection of various VOCs through their cognate ORs. The electrophysiological signals corresponding to the binding of VOCs with the ORs are detected on a monitor. Due to the creation of small channels around the droplet via hydrophobic force, the system has precision similar to insect ORs and can detect the presence of molecules at the sensitivity level of one part per billion, making it sensitive enough to detect VOCs from oral microbiome.

### Example 2

A research study will be conducted in the form of a randomized clinical trial. Participants with varying diets and genetic backgrounds must be selected so that the link between oral microbes and diabetes is of wider applicability. Each participant will use our device periodically and the oral microbiota in each participant will be determined. The prediction of diabetes will be tested in the study participants. Based on this, the links between oral microbes and diabetes can be confirmed and the dietary/lifestyle information of participants can be collected so that in clinical practice, proper preventative action can be taken upon prediction.

### REFERENCES

All references, including granted patents and patent application publications, referred herein are incorporated herein by reference in their entirety.
Aas J A, Paster B J, Stokes L N, Olsen I, Dewhirst F E. Defining the normal bacterial flora of the oral cavity. J Clin Microbiol. 2005; 43:5721.
Wade W G. The oral microbiome in health and disease. Pharmacol Res. 2013; 69:137.
Long J, Cai Q, Steinwandel M, Hargreaves M K, Bordenstein S R, Blot W J, Zheng W, Shu X O. Association of oral microbiome with type 2 diabetes risk. J Periodontal Res. 2017 June; 52(3):636-643.
Chen B, Wang Z, Wang J, Su X, Yang J, Zhang Q, Zhang L. The oral microbiome profile and biomarker in Chinese type 2 diabetes mellitus patients. Endocrine. 2020 June; 68(3):564-572.
Choudoir M, Rossabi S, Gebert M, Helmig D, Fierer N. A Phylogenetic and Functional Perspective on Volatile Organic Compound Production by Actinobacteria. mSystems. 2019 Mar. 5; 4(2): e00295-18.
Yamada T, Sugiura H, Mimura H, Kamiya K, Osaki T, Takeuchi S. Highly sensitive VOC detectors using insect olfactory receptors reconstituted into lipid bilayers Sci Adv. 2021 Jan. 13; 7(3):eabd2013.
Seneviratne C J, Balan P, Suriyanarayanan T, Lakshmanan M, Lee D, Rho M, Jakubovics N, Brandt B, Crielaard W, Zaura E. Oral microbiome-systemic link studies: perspectives on current limitations and future artificial intelligence-based approaches. Crit Rev Microbiol. 2020 May; 46(3): 288-299.
Marianne Defresne M, Barbe S, Schiex T. Protein Design with Deep Learning. Int J Mol Sci. 2021, 29; 22(21): 11741.
Cheema J A, Carraher C, Plank N O V, Travas-Sejdic J, Kralicek A. Insect odorant receptor-based biosensors: Current status and prospects. Biotechnol Adv. 2021 December; 53:107840.
Verbrugghe P, Brynjolfsson J, Jing X, Bjorck I, Hallenius F, Nilsson A. Evaluation of hypoglycemic effect, safety and immunomodulation of Prevotella copri in mice. Sci Rep. 2021 Oct. 28; 11(1):21279.
Luo J, Zhang H, Lu J, Ma C, Chen T. Antidiabetic effect of an engineered bacterium Lactobacillus plantarum-pMG36e-GLP-1 in monkey model. Synth Syst Biotechnol. 2021 Sep. 17; 6(4):272-282. JP2020091120A
KR101940445B1
US20190227044A1

What is claimed is:

1. A device comprising a component comprising a wall having an outer layer and an inner layer, wherein the inner layer comprises a hydrophobic layer comprising an unit having a lipid layer surrounding an environment comprising an aqueous environment, wherein the lipid layer comprises an olfactory receptor configured to capture one or more volatile organic compounds (VOCs), wherein the device is configured to detect one or more VOCs.

2. The device of claim 1, wherein capture of one or more VOCs is configured to produce a signal.

3. The device of claim 1, wherein the unit and the hydrophobic layer of the inner layer interact using a hydrophobic bond.

4. The device of claim 3, wherein the hydrophobic bond is configured to form a space between the unit and the hydrophobic layer such that the space is configured to allow passage of the one or more VOCs to the olfactory receptor present on the unit.

5. The device of claim 4, wherein the device is configured to detect one or more VOCs with a sensitivity within a range of about one part per million to about one part per billion.

6. The device of claim 5, wherein the sensitivity is about one part per billion.

7. The device of claim 4, wherein one or more VOCs comprise molecules released from a microbiome of a user using the device.

8. The device of claim 7, wherein the microbiome comprises gut microbe and/or oral microbiome.

9. The device of claim 7, wherein the microbiome includes Actinobacteria and/or Prevotella.

10. The device of claim 1, wherein the lipid layer comprises a lipid bilayer.

11. The device of claim 1, wherein the component comprises a slit comprising a microslit having the unit.

12. The device of claim 7, wherein the device is configured to detect onset of a diabetes.

13. A method to detect one or more volatile organic compounds (VOCs) comprising:
entering of one or more VOCs into a slit of a device;
attaching one or more VOCs to a unit having a lipid layer surrounding an environment comprising an aqueous environment, wherein the lipid layer comprises an olfactory receptor;
capturing of the one or more VOCs by the olfactory receptor;
generating one or more ionized molecule from the capture of one or more VOCs; and
producing an electrophysiological signal.

14. The method of claim 13, wherein one or more VOCs comprise one or more microbial VOCs.

15. The method of claim 13, wherein the method further comprises analyzing the VOCS by employing a deep learning algorithm.

16. The method of claim 13, wherein the method is configured to detect a diabetic condition of an user.

17. A system comprising an artificial olfactory sensing system comprising:
a unit having a lipid layer surrounding an environment comprising an aqueous environment, wherein the lipid layer comprises an olfactory receptor;
a transistor to capture an ionized molecule to generate an electrophysiological signal;
a monitor to display the electrophysiological signal in an quantitative and an qualitative manner;
wherein the system is configured to detect one or more Volatile Organic Compounds (VOCs).

18. The device of claim 17, wherein the VOCs comprises at least one of (S)-2-hydroxypropanoic acid, heptylhydroperoxide, 2,3-dihydroxypropanal, nonanoyl chloride, dodecanal, (Z)-2-nonenal, 4,5-dimethyl, -3(2H)-isoxazolone, (Z)-2-decenal, trichloro acid 3-tridecyl ester, levoglucosan, 4-(dimethyl amino)-3-methyl-2-butanone, 4-methyl-1-butene-1, 1-pentanoic acid ester, diethylphthalic acid, 1-chloro-8-heptadecene, pentadecanoic acid, 1,2-benzenedicarboxylic acid butyldecyl ester, nonanal, 1-butanol, 3-hydroxy-2-butanone, hexanol, 2-pentanone, tetrahydrofuran, 2-methylpyrazine, (E)-2-nonenal.

19. The device of claim 17, wherein the ionized molecule is negatively charged.

20. The device of claim 17, the system comprises a deep learning algorithm configured to detect one or more VOCs.
